(19) United States
(12) Patent Application Publication
(10) Pub. No.: US 2024/0260948 A1
(43) Pub. Date: Aug. 8, 2024

(54) Microneedle-Mediated Wearable Dermal Patch and Methods of Use for Collection of Biological Fluid

(71) Applicant: Curiva, LLC, Goshen, IN (US)

(72) Inventors: Eydis Lima, Goshen, IN (US); Tracie MCGINNITY, Chicago, IL (US)

(73) Assignee: Curiva, LLC, Goshen, IN (US)

(21) Appl. No.: 18/291,357

(22) PCT Filed: Jul. 22, 2022
(86) PCT No.: PCT/US22/38092

§ 371 Date: Jan. 23, 2024

Related U.S. Application Data
(60) Provisional application No. 63/225,169, filed on Jul. 23, 2021.

(51) Int. Cl. A61B 10/00; A61B 5/103; A61B 5/145; A61B 5/1459

(52) U.S. Cl. CPC A61B 10/0045; A61B 5/1032; A61B 5/14514; A61B 5/1459; A61B 10/0041; A61B 2010/008

(57) ABSTRACT

The present invention provides dermal patches and methods of use for collecting biological fluid in proximity to a lymph node for ex vivo or in situ detection of a trigger analyte. In one embodiment, the invention provides a method for collecting a biological fluid sample, the method comprising: a) applying a dermal patch comprising a microneedle array to a skin location in proximity to at least one lymph node in a subject or patient; b) contacting the microneedle array intradermally to access a biological fluid; c) allowing the microneedle array to take up a biological fluid sample; d) collecting the biological fluid sample into a sampling chamber housed within the dermal patch. Additional steps can include activating the microneedle array by applying pressure or electric current.

### CROSS REFERENCE TO RELATED APPLICATIONS

This application claims priority to U.S. Provisional Appl. No. 63/225,169 filed Jul. 23, 2021, which is incorporated by reference herein in its entirety.

### BACKGROUND OF INVENTION

To date, no rapid and/or non-invasive diagnosis for women's cancer, such as cervical, endometrial, and ovarian cancer exists. Traditional tests involve drawing blood from the patient, sending the blood sample to a lab, and waiting for results. Current diagnostic practice may also include tumor biopsy, which is even more invasive and similarly requires lab time and equipment. Moreover, a tumor biopsy may be hindered by the location and surgical accessibility of the target tissue site. The discomfort and anxiety of the blood draw and/or biopsy procedure, as well as delays in obtaining results, discourage people from testing, follow-up, and compliance altogether. Because of the lack of testing options, patients are susceptible to high risks of advanced cancer stages, infertility, not being able to bear children, and poor quality of life if afflicted impacting the years lived with a disability (YLDs) due to prevalent cases of the disease.

The present invention provides a less invasive and less painful biological fluid collection process by using a microneedle-mediated dermal patch. The collection of biological fluids from microneedle-mediated dermal patch is known in the art. But current microneedle-mediated dermal patches are generally applied and designed to collect biological samples of systemic or circulating biomarkers from blood, plasma, or interstitial fluid. See, e.g., Ripolin A et al. “Successful application of large microneedle patches by human volunteers” (2017) Int'l J Pharm 521(1-2):92-101.

Some of the methods and devices described herein are applied and designed to collect lymph fluid, which contains biomarkers that may be localized near a site of malignancy. A microneedle-mediated wearable pre-analytical or wearable diagnostic patch for use in collecting localized fluid samples and detecting trigger analytes, particularly of gynecologic malignancies, is of utmost need in the clinical workflow for improvements in supporting monitoring and real-time risk guidance for earlier stages surgeries along with treatment plans.

### BRIEF SUMMARY OF THE INVENTION

In one embodiment, the invention provides a method for collecting a biological fluid sample, the method comprising: a) applying a dermal patch comprising a microneedle array to a skin location in proximity to at least one lymph node in a subject or patient; b) contacting the microneedle array intradermally to access a biological fluid; c) allowing the microneedle array to take up a biological fluid sample; d) collecting the biological fluid sample into a sampling chamber housed within the dermal patch. Additional steps can include activating the microneedle array by applying pressure or electric current.

In preferred embodiments, the method further comprises detecting a trigger analyte in the biological fluid sample. Detection can be accomplished by PCR, ELISA, LAMP, or synthetic riboregulator detection. Additional steps can include emitting a colorimetric or fluorescent detection signal when a trigger analyte is detected in the biological fluid sample.

In some embodiments, the lymph node is a cervical, clavical, pectoral, axillary, brachial, mediastinal, hilar, spleen, paraaortic, mesenteric, iliac, inguinal, femoral, or popliteal lymph node. In certain embodiments, the paraaortic, mesenteric, or iliac lymph node is preferred. Similarly, certain lymph nodes can be targeted by applying the dermal patch to the pelvic region, particularly for female patients. In this case, the trigger analyte for detection is correlated with a gynecological malignancy.

Other embodiments of the invention describe a wearable dermal patch for use in detecting trigger analytes in a biological fluid sample, comprising: a) at least one microneedle array (for example, comprising hydrogel microneedles) dimensioned to access a biological fluid at a location in proximity to at least one lymph node; b) a sample inlet in fluid communication to the microneedle array; c) at least one microchannel positioned to provide fluid communication between the sample inlet and at least one detection microwell, wherein each detection microwell comprises a detection reagent configured to activate and emit a detection signal when the detection reagent contacts a trigger analyte (e.g., an oncoprotein or miRNA), and wherein the trigger analyte correlates to the lymph node. In some embodiments, the dermal patch further comprising a control microwell, wherein the control microwell comprises a control reagent configured to activate and emit a control signal when the control reagent contacts a biological fluid. The detection signal and the control signal can be independently colorimetric or fluorescent.

In one embodiment, the dermal patch accepts a sample volume of 10-1000 μL.

### BRIEF DESCRIPTION OF THE DRAWINGS

FIG. 1A shows hollow microneedles (100) inserted through layers of the skin (i) stratum corneum, (ii) epidermis, and (iii) dermis. FIG. 1B shows the direction (arrow) of biological fluid sample uptake through the hollow microneedles.

FIG. 2 shows an exemplary layout of microfluidic detection. The arrow shows the inward flow direction of sample from a microneedle array (not pictured) to a microfluidic system sampling chamber (220). The preliminary storage well (230) provides necessary modifications of the sample type to then proceed onwards to a first (241), second (242), and third (243) detection microwell. The detection microwells house freeze-dried detection reagents, e.g. toehold switch sensors, to detect trigger analytes and emit a detection signal if trigger analytes are present. An excess fluid storage well (232) stores excess sample volume not required for detection. In one embodiment, thickness (t) of the microfluidic system sampling chamber is 0.1 to 0.9 mm to account for incorporation into the other patch layers (not shown).

FIG. 3 shows exemplary layers for constructing a single detection diagnostic dermal patch according to one embodiment. A posterior layer (350) (outermost layer, visible after application) includes an adhesive on the anterior face (towards the skin, arrow). A single detection window (351) displays a detection signal only when a trigger analyte is detected. A microfluidic layer (360) shows upper views of first and second storage wells (334a, 335a) and upper views of a single detection microwell (341a). First and second microneedle arrays (311, 312) are in fluid communication with first and second storage wells, respectively (334, 335) for sample acquisition hold. Dimensions for this embodiment of the dermal patch can be 5 cm×5 cm×1 mm.

FIG. 4 shows one embodiment of the layers and assembly configuration of the dermal patch with multiple detection microwells. A posterior (top) layer (450) includes an adhesive anterior face. First, second, and third detection windows (451, 452, 453) correspond to different trigger analytes, wherein a detection signal is only emitted when the trigger analyte is detected. Items (434a, 435a) indicate the upper views of first and second storage wells, respectively. Items (441a, 442a, 443a) indicate the upper views of first, second, and third detection microwells, respectively, for separate analytes. First and second microneedle arrays (411, 412) are housed on an anterior (skin-facing) layer (470), which protects dermal patch components from the environment. Dimensions for this embodiment of the dermal patch can be 8 cm×5 cm×1 mm, with the length of the detection section (windows collectively) to be 4 cm.

FIG. 5 shows a suggested patch application location on the pelvic region of a female patient.

FIG. 6 shows exemplary layers for constructing a diagnostic dermal patch according to one embodiment. FIG. 6A shows the posterior layer, which is visible after dermal application. FIG. 6B shows the microfluidic layer. FIG. 6C shows the anterior (skin-facing) layer.

FIG. 7 shows the relative arrangement of the posterior, microfluidic, and anterior layers in an exemplary wearable diagnostic dermal patch assembly, and features a raised indicator (722) for microneedle activation.

A summary of reference numbers and reference items is provided. Different views of same features are indicated by (a). The designation of “first,” “second,” and “third,” is for labeling clarity only and does not require positioning or temporal order.

| Ref. No. | Ref. Item |
|---|---|
| | Microneedles and Sample Uptake |
| 10 | microneedle |
| 11 | 1st or single microneedle array |
| 12 | 2nd microneedle array |
| 20 | microfluidic system sampling chamber |
| 22 | microneedle activation indicator |
| 25 | sample inlet |
| 27 | microfluidic channel |
| | Storage wells |
| 30 | storage well |
| 32 | storage well (excess) |
| 34 | first storage well |
| 35 | second storage well |
| | Microwells |
| 41 | 1st or single detection microwell |
| 42 | 2nd detection microwell |
| 43 | 3rd detection microwell |
| 45 | control microwell |
| | Layers |
| 50 | posterior (visible after application) layer |
| 60 | microfluidic layer |
| 70 | anterior (skin-facing) layer |
| | Windows |
| 51 | 1st or single detection window |
| 52 | 2nd detection window |
| 53 | 3rd detection window |
| 55 | control window |

### DETAILED DESCRIPTION OF THE INVENTION

The present invention provides methods for collecting a biological fluid sample, particularly a lymph fluid sample, using a microneedle-mediated dermal patch. The methods can further comprise a detection step, wherein a trigger analyte in the collected fluid sample is detected ex vivo (separately from the patch, e.g., by lab-based analytical techniques) or in situ (integral to the dermal patch within a detection microwell). The present invention also provides microneedle-mediated dermal patches for collecting a biological fluid sample, and optionally additionally detecting trigger analytes in the biological fluid sample in situ.

### I. Definitions

As used herein, “lymph nodes” refer to any lymph nodes present in the subject (animal model) or patient (human) body. The human body contains about 450 lymph nodes, concentrated in location near the trunk of the body. Lymph nodes include, but are not limited to cervical (neck), clavical, axillary (under arm), pectoral, axillary, brachial, mediastinal, hilar, spleen, paraaortic (ovaries/testes), mesenteric, iliac, inguinal (groin), femoral, and popliteal lymph nodes. Lymph nodes include both deep tissue and surface tissue lymph nodes.

As used herein, the term “biological fluid sample” refers to any biological fluid collected via transdermal application of a microneedle array. The biological fluid sample may include blood, plasma, interstitial fluid, lymph fluid or combinations thereof.

The term “lymph fluid” refers to biological fluid obtained from a location in proximity to at least one lymph node. Without being bound by theory, it is believed that fluid samples collected from certain sites exhibit a high concentration of localized biomarkers and/or accumulated exosomes released from adjacent sites of malignancy. Anatomical reservoirs of lymph fluid include lymph nodes and lymph vessels. While a lymph fluid sample may be collected directly from lymph nodes or lymph vessels (e.g., by directly contacting the microneedles to a lymph node or lymph vessel), collection of a lymph fluid sample from a location in proximity to at least one lymph node acceptably exhibits the localized biomarkers.

As used herein, the term “microneedle array” refers to a plurality of microneedles that can project perpendicularly from the dermal patch to pierce through dermal layers and access biological fluid reservoirs.

As used herein, the term “dermal patch” refers to a substrate for supporting and actuating the microneedle array. The substrate can be made of any suitable and dermally compatible, preferably hypo-allergenic, substance. The dermal patch includes an anterior surface which directly faces the dermal skin application location and a posterior surface which may include indicators for the regions of actuating the microneedle array and/or for reporting detection in situ. As is known in the art, the dermal patch can optionally include an adhesive layer disposed in the same direction as the microneedle array for affixing the patch to the skin during sample collection and/or detection.

As used herein, the term “trigger analyte” refers to any biomarker detectable in a fluid sample. The trigger analyte can be a protein, DNA, or RNA molecule. In preferred embodiments, the trigger analyte is an oncoprotein or miRNA. In preferred embodiments of the present invention, the trigger analyte(s) are selectively correlated to the localized source of sample collection. For example, when lymph fluid is collected from the pelvic region of a female patient, the trigger analyte is selected from those correlated to a gynecological malignancy.

The term “gynecological malignancy” refers to any pre-cancerous or cancerous, benign or malignant growths or tumor state of female reproductive organs, particularly the cervix, ovaries, uterus, endometrium. Gynecological malignancies include, but are not limited to: cervical cancer, ovarian cancer, endometriosis-associated ovarian cancer, endometrial cancer, endometriosis, and fibroids.

### II. Methods for Collecting a Biological Fluid Sample by Microneedle-Mediated Dermal Patch

In one embodiment, the present invention provides methods and devices for collecting a biological fluid sample by a microneedle-mediated dermal patch. When the dermal patch is used solely for fluid sample collection (e.g., for ex vivo detection), we refer to this embodiment as a pre-analytical dermal patch. When detection and reporting steps are integrated onto the dermal patch (e.g., for in situ detection), we refer to this embodiment as a diagnostic dermal patch.

One of ordinary skill in the art would recognize known mechanical and biological properties to be achieved by microneedle-mediated dermal patches. See, e.g., Coffey J W et al 2013 “Early circulating biomarker detection using a wearable microprojection array skin patch” Biomaterials 34(37): 9572-83; Samant P P 2018 “Mechanisms of sampling interstitial fluid from skin using a microneedle patch” PNAS 115(18): 4583-88; Corrie et al. U.S. Pat. No. 9,387,000; Miller P R et al. 2018 “Extraction and biomolecular analysis of dermal interstitial fluid collected with hollow microneedles” Commun Biol. 1:173; Paranjape WO 2018/144506.

In one embodiment, the present invention provides a dermal patch for collecting a biological fluid sample, particularly a lymph fluid sample. The dermal patch comprises at least one microneedle array and a sampling chamber in fluid connection to the microneedle array.

The microneedle array can comprise solid, hollow, or hydrogel microneedles or a combination thereof. In particular embodiments, the microneedle array comprises hydrogel microneedles. Exemplary microneedle arrays, including those applied by dermal patches, are known to one of ordinary skill in the art. See, e.g, Ripolin 2017. In one embodiment, the microneedles are dimensioned to access biological fluid in proximity to a lymph node. The depth of insertion (e.g., microneedle length) can be 300-600 μm, 400-600, or about 300, 400, or 500 μm. As is known in the art, it is desirable for the maximum insertion depth to be no greater than about 600 μm to minimize or avoid contact with pain receptors. In one embodiment, the microneedles are 600 μm tall, with a base width of 300 μm, and cover a 0.5 cm² area and are in a 19×19 microneedle array.

The microneedle array can present in a fixed-actuated state whereby contacting the dermal patch to the skin inherently contacts the microneedle array to a biological fluid. Alternatively, the microneedle array can include both a passive and active state, whereby the microneedle array does not inherently contact the biological fluid when the dermal patch is applied to the skin. Rather after the dermal patch is first applied to the skin, then the microneedle array can be subsequently activated, e.g., by applying pressure, electric current, or other means.

The dermal patches of the present invention can include a single microneedle array or two or more arrays on the same dermal patch. When two or more arrays are employed, each may be fluidly connected to the same sampling chamber (to aggregate the fluid samples collected from multiple arrays), or each array may be fluidly connected to separate sampling chambers (to isolate the fluid samples collected from multiple arrays). The microneedle arrays can be the same as or different from one another. For example, microneedle depth may be varied to access variable depths of lymph nodes, or to otherwise access different biological fluid reservoirs. Likewise, the microneedles can vary in dimension or construction within a single array.

In a preferred embodiment, the dermal patch is applied to a skin location in proximity to at least one lymph node, preferably a plurality or cluster of lymph nodes. Proximity to the lymph node can be measured as the distance from the distal tip of the microneedle array to the surface of the nearest lymph node. The proximity is preferably less than 10, 9, 8, 7, 6, 5, 4, 3, 2, 1, or 0.5 cm. Accordingly, in instances of direct lymph fluid collection, where fluid is taken up directly by the microneedle array from a lymph node or vessel, the proximity equals 0 mm. In one embodiment, the skin location overlies the lymph node or nodes, such that proximity equals the depth of the lymph node from the epidermis surface minus the length of the microneedles. It is understood that exact alignment overlying the lymph nodes and/or direct collection from lymph nodes or vessels is not required to achieve the purpose of this invention, so long as the microneedle array contacts a source of localized biomarkers. Application of the dermal patch to a suitable location to collect lymph fluid can be performed by using approximate bodily regions of lymph node anatomy known to one of ordinary skill in the art. Optionally, the location precision relative to lymph nodes can be enhanced by palpating the target lymph nodes, and/or by employing one or more standard techniques for lymph node imaging, such as ultrasound, CT, MRI, or PET.

Lymph nodes include, but are not limited to cervical (neck), clavical, axillary (under arm), pectoral, axillary, brachial, mediastinal, hilar, spleen, paraaortic (ovaries/testes), mesenteric, iliac, inguinal (groin), femoral, and popliteal lymph nodes. Lymph nodes include both deep tissue and surface tissue lymph nodes.

In certain embodiments, the skin location for applying the dermal patch can be a neck, underarm, or pelvic region. In one embodiment, the dermal patch is applied to the pelvic region.

The dermal patch houses a sampling chamber to accept the fluid sample taken up by the microneedle array. The fluid sample moves from the microneedle array to the sampling chamber by a sample inlet.

In one embodiment, the sampling chamber comprises one or more storage wells that store the collected fluid sample. A storage well can be empty (without reagents) or may include one or more reagents, e.g., to stabilize the collected sample or prepare the collected sample for analysis. The collected fluid sample can then be extracted from the dermal patch for ex vivo detection. Alternatively, the collected fluid sample can proceed via microfluidic channel to detection microwells.

In one embodiment, the sampling chamber is a microfluidic system having one or more microwells and one or more microfluidic channels to connect the sample inlet to the microwells. The microwells can be used to analyze or store collected fluid sample. For example, microwells can contain detection or control reagents that activate upon contact with the collected fluid sample. The microfluidic system can also include one or more storage wells that precede (e.g., for sample preparation) or follow (e.g., for storage of excess fluid sample) the detection microwells in fluid direction from the sample inlet.

The sampling chamber can accept a sample volume of 10-1000 μL, preferably 50-500 μL, or about 50 μL, about 250 μL, or about 500 μL. This sampling chamber volume does not include the residual fluid sample volume residing on or in the microneedles after collection. Depending on the quantity and type of microneedles, and the type of uptake employed (e.g., passive, current-driven, etc.), one of ordinary skill in the art could readily determine the appropriate duration of collection time between the microneedles and biological fluid source.

The methods can include a step of allowing the microneedle array to take up a biological fluid sample. This collection time is measured starting from the activation of thea microneedles (e.g., from the transdermal injection of the microneedles not necessarily the application of the dermal patch) until the target sample volume is collected (e.g., about 50 μL, about 250 μL, or about 500 μL). In some embodiments, the collection time is 5 to 360 minutes, 5 to 180 minutes, 60 to 120 minutes, 10 to 60 minutes, less than 120 minutes, less than 60 minutes, or about 10, 15, 20, 25, 30, 45 or 60 minutes. In other embodiment, the collection time is 1-24 hours, 1-12 hours, 12-24 hours, 1-8 hours, 2-4 hours, or 1-2 hours.

For both pre-analytical dermal patch embodiments and diagnostic dermal patch embodiments, the dermal patches of the present invention can further include one or more one-way valves to permit unilateral direction of fluid flow. Valves can be employed, for example to prevent fluid from the sampling chamber back to the microneedles array, and/or to direct the flow of fluid within a microfluidic system. Similarly, pressure-activated blisters can be employed to control the direction and volume of fluid flow between wells on the dermal patch.

For both pre-analytical dermal patch embodiments and diagnostic dermal patch embodiments, the dermal patches of the present invention can further include an anterior layer comprising an adhesive disposed in the same direction as the microneedle array to assist in maintaining contact during fluid collection. Dermal contact can be optionally maintained but is not required during the detection phase for diagnostic dermal patches.

For both pre-analytical dermal patch embodiments and diagnostic dermal patch embodiments, the dermal patches of the present invention can further comprise a control microwell comprising a control reagent that emits a visible (e.g., colorimetric or fluorescent) control signal when the control reagent contacts the biological fluid sample to verify successful uptake of the targeted biological fluid.

### III. Methods for Detecting a Trigger Analyte

In some embodiments, the methods and devices of the present invention comprise a detection step comprising detecting a trigger analyte in the biological fluid sample ex vivo or in situ. Regardless of whether detection is performed ex vivo (e.g., separate from the dermal patch or lab-based) or in situ (e.g., integral to the dermal patch, e.g., in a detection microwell), features of trigger analyte selection and detection methodology are described below.

The trigger analyte can be a protein, DNA, or RNA molecule. In preferred embodiments, the trigger analyte is a protein (e.g., an oncoprotein) or RNA (e.g., a miRNA).

In certain embodiments, the trigger analyte is selected to correspond to the intended patch application location, more specifically to the lymph nodes in proximity to the patch application location. For example, the dermal patch can be applied in proximity to a paraaortic and/or iliac lymph node, and the trigger analyte to be detected can include a biomarker correlated to a gynecological malignancy. A similar approach could be employed for testicular malignancies. As another example, the dermal patch can be applied in proximity to an axillary or pectoral lymph node, and the trigger analyte to be detected is correlated with breast malignancies. The dermal patch can be applied in proximity to a cervical (neck) lymph node, and the trigger analyte to be detected is correlated to thyroid malignancies.

In other embodiments, the skin location and trigger analytes are intentionally selected to not be physiologically correlated. For example, testing lymph fluid from a lymph node distant from the site of malignancy may report diagnostically useful information about cancer stage or metastasis.

In specific embodiments, particularly those where the dermal patch is applied to the pelvic region and the subject/patient is female, the trigger analyte is a biomarker associated with a gynecological malignancy. In specific embodiments, the trigger analyte is correlated to cervical cancer, ovarian cancer, endometriosis-associated ovarian cancer, endometrial cancer, endometriosis, or fibroids.

The subject or patient can be any sex. In some embodiments, the trigger analyte is selected to correlate to the subject/patient sex. In one embodiment, the subject/patient is female. In another embodiment, the subject/patient is male.

In one embodiment, the trigger analyte is a miRNA sequence upregulated in HPV. In other embodiments, the trigger analyte is a miRNA sequence significantly overexpressed in other stages of cervical cancer such as cervical intraneoplasias I-III (CINI-CINIII). Exemplary respective oncoproteins or miRNA sequences for certain gynecological malignancies are listed in Table 1 below. These and other trigger analytes would be readily recognized by one of ordinary skill in the art. See, e.g., Huang J et al., J Med Genet (2019) 56: 186-194; Jia W et al, Molecular and Clinical Oncology (2015) 3:851-858; Babion I et al., Clinical Epigenetics (2018) 10:76; Park S et al., BMC Cancer (2017) 17:658; Nagamitsu Y et al., Molecular and Clinical Oncology (2016) 5:189-194; Xin F et al., Eu Rev Med Pharmacol Sci (2016) 20:4846-4851; Zavesky L et al., Neoplasma (2016) 62(4): 509-520; Zhang L et al, International Journal of Gynecological Cancer (2016) 26(5): 810-816; Zhang W et al., International Journal of Oncology (2019) 54: 1719-1733; Fan Y et al., Disease Markers (2018) Article ID 4826547; Pedroza-Torres A et al., Gynecologic Oncology (2016) 142: 557-565; Han M S et al., Biomed Research International (2018) Article ID 1942867; Shen Y, Xu Q X, Annals of Oncology (2018) 29(S9): 267P; Ratnam S et al., Journal of Clinical Microbiology (2011) 49(2): 557-564; Prahm K P et al., PLOS ONE (2018) 13(11): e0207319; Li R N et al., Oncology Letters (2017) 13: 4397-4401.

TABLE 1
Target biomarkers consisting of driver mutations/oncoproteins and miRNA sequences that can be
used as the trigger analyte. Concentrations range from 2-500 ng in blood or interstitial fluid.

| | Disease | |
|---|---|---|
| Disease Type | Stage | Target Biomarkers |
| Cervical cancer | CIN I- | PTBP3, ESX1, PER3, CIP2A, HPV16, HPV18, HPV33, HPV58* |
| | III | |
| Cervical cancer | CIN I- | miR-21, miR-25, miR-29a, miR-200a, miR-486** |
| | II | |
| Cervical cancer | CIN | miR-15b-5p, miR-26a, miR-29a, miR-99a, miR125b-5p, miR145, miR149-5p, |
| | III | miR203, miR203a-3p, miR-372, miR-375, miR-513** |
| Cervical cancer | HPV | HPV-6, HPV-11, HPV-16, HPV-18, HPV-26, HPV-31, HPV-33, HPV-34, |
| | 16 | HPV-35, HPV-39, HPV-40, HPV-42, HPV-44, HPV-45, HPV-51, HPV-52, |
| | E6/E7 | HPV-53, HPV-54, HPV-56, HPV-58, HPV-59, HPV-61, HPV-62, HPV-66, |
| | | HPV-68, HPV-69, HPV-70, HPV-71, HPV-72, HPV-73, HPV-81, HPV-82, |
| | | HPV-83, HPV-84, HPV-89* |
| | | miR-15a, miR-15b, miR-16-1, miR-22-29a, miR-23b, miR-25-92a, miR-29, |
| | | miR-34a, miR- 146a, miR-148a-3p, miR-190a-5p, miR-199b- 5p, miR-203, |
| | | miR-218, miR-324-5p** |
| Ovarian cancer | — | CA125, BRCA1, BRCA2, IMP1* |
| | | miR-30c, miR-92, miR-93, miR-126, miR-127, miR-130a, miR-155, miR- |
| | | 335, miR-181a, miR-21, miR-141, miR-200a, miR-200b, miR- 200c, miR- |
| | | 205, miR-214, miR-996** |
| Serous ovarian | — | miR-16, miR-191, miR-4284** |
| cancer | | |
| Endometriosis- | — | miR-16, mi-21, miR-132, miR-191, miR-362-5p, miR-628-3p, miR-1915, |
| associated | | miR-1975** |
| ovarian cancer | | |
| (EAOC) | | |
| Endometrial | — | miR-17-92, miR-30c, miR-194, miR-200c, miR-205** |
| cancer | | |
| Endometriosis | — | LUM, CPM, TNC, TPM2, TPM3 PAEP, CA-15-3, CA-19-9, CA-125, |
| | | slCAM1, FST, VEGF, MCP-1, MIF, IL-1, IL1R2, IL-6, IL8, IL-10, IL-12, IL- |
| | | 17, IL-23, Annexin V, glycodelin, TNF-alpha, laminin-1, INF-gamma, CD40, |
| | | CD40L, ADAM8, RANTES, osteopontin, MMP-2, MMP-3, |
| | | MMP-9, PEDF, c-Met, FGF-2, angiogenin, VEGFR-1, cardiolipin, PDIK1L, |
| | | SLP2, TMOD3, FOXP3+* miR-9, miR-16, miR-122, miR-141, miR-145, |
| | | miR-191, miR-195, miR-199a, miR-542-3p** |

*driver mutations/oncoproteins
**miR

Methods of detecting trigger analytes (e.g., biomarkers) are well known in the art including, but not limited to: PCR, ELISA, LAMP, or detection by synthetic riboregulator (e.g., toehold switch). In one embodiment, the detection method is ex vivo (lab-based and/or bench-top point of care). In another embodiment, the detection method is in situ (integral to the dermal patch, that is, detection occurs within a nanosensing chamber within the dermal patch).

Especially when the detection step is conducted in situ, the methods and devices can further comprise a reporting step, wherein a visible (e.g., colorimetric or fluorescent) reporting signal is produced when the trigger analyte is detected. In this case, the diagnostic dermal patch can include one or more posterior viewing windows for reporting the detection signal (or alternatively may be wholly posteriorly transparent). A similar posterior viewing window may be employed for reporting a control signal to verify successful sample collection. Posterior (outward-facing) indicators can help the user distinguish between one or more detection windows or between a detection window and a control window.

In situ detection is highly desirable to provide rapid (ideally same-day or same-visit) detection results to facilitate rapid diagnosis. By incorporating at least one detection microwell onto the dermal patch, the detection reagents present therein can detect and report the presence of trigger analytes in the collected biological sample in 24 hours or less, preferably equal to or less than 12, 8, 6, 4, 3, 2, 1, or 0.5 hours, including sample collection time (e.g., measured starting from the activation of the microneedles).

### IV. Diagnostic Dermal Patch Using Toehold Switch

In one embodiment, the present invention provides a diagnostic dermal patch for both collecting a biological fluid sample and detecting one or more trigger analytes. A diagnostic dermal patch comprises: at least one microneedle array; a sampling chamber connected to the microneedle array; at least one microchannel in fluid communication between the sampling chamber and at least one nanosensing chamber; each nanosensing chamber comprising at least one detection microwell, wherein each detection microwell comprises a synthetic riboregulator (e.g., a toehold switch) configured to activate and emit a reporting signal (e.g., colorimetric or fluorescent signal) when the synthetic riboregulator contacts the trigger analyte.

The diagnostic dermal patch described herein can detect a panel of oncoproteins and miRNAs present and significantly upregulated within the tissues of individuals with cervical cancer during both the cervical intraneoplasias (CINs) pathological states and the precursor human papillomavirus (HPV) stage. The dermal patch utilizes microneedles to painlessly and transdermally extract a biological fluid from the patient. The biological fluid can be plasma, interstitial fluid, or preferably lymph fluid. Then, a detection reagent (such as synthetic riboregulators, e.g., toehold switches) present within microwells of the nanosensing chamber can interact with the collected sample fluid, and upon recognition of the trigger analyte, e.g., an oncoprotein or RNA, will express a detection signal to indicate the presence of the targeted disease stage. This wearable diagnostic patch can provide highly accurate, pain-free testing and possible digitization for real-time monitoring for better care within those afflicted in the gynecologic health sector.

In one embodiment, the dermal patch comprises a single diagnosing chamber. The diagnosing chamber comprises a nanosensing chamber, and an outer adhesive protective layer. The nanosensing chamber includes one or more microwells housing freeze-dried synthetic riboregulators toehold switches to recognize oncoproteins/RNA molecules that are disease-associated biomarkers, also referred to herein as trigger analytes.

One of ordinary skill in the art would also recognize known methods for detecting analytes using synthetic riboregulators, including “hairpin” riboregulators and/or toehold switches. See, e.g., Green A A et al. 2014 “Toehold Switches: De-Novo-Designed Regulators of Gene Expression” 159(4): 925-39; Han et al. U.S. Pat. No. 9,725,715; Isgut U.S. Pat. No. 10,041,109; Kim et al. WO 2017/087530; Yin et al. U.S. Pat. No. 9,217,151.

Toehold switches in the microwells of the diagnosing chamber sense the presence of the target infection or disease by reacting with the oncoprotein or RNA molecules that indicate the existence of such infections or diseases. Toehold switches are designed prokaryotic riboregulators that seek to activate gene expression signals in the presence of analogous RNAs with selective sequences to infectious agents and diseases. Synthetic riboregulators take advantage of targeting the sequence around the start codon to repress protein translation, avoiding any base pairing to the ribosome binding site (RBS) or start codon itself to regulate translation. Because of this feature, the toehold switch can be designated to activate in response to a trigger oncoprotein or RNA with the specific matching sequence, enabling translation of a signal-expressing protein.

The toehold switch mechanism functions as follows: the inactive toehold switch lies dormant in a hairpin configuration, which disables protein translation by sequestering the RBS and start codon. When the customizable toehold switch interacts with a trigger oncoprotein or RNA, the toehold switch becomes linear, revealing the RBS and start codon and activating protein translation of the signaling protein or molecule of interest. The toehold switch is customizable to activate on only with the presence of a targeted, trigger oncoprotein or RNA associated with a targeted infection or disease.

The toehold switches can be customized to work with any one of numerous oncoproteins or miRNA sequences, including the ones described herein.

In one embodiment, the toehold switches in the diagnosing chamber are housed in a freeze-dried form. Freeze-drying preserves structure and functionality, which allows for diagnostic development with signal read-outs. The physical characteristics of these freeze-dried toehold switches allow them to be incorporated into a convenient diagnostics dermal patch. When the sample, such as blood, interstitial fluid, or lymph, is introduced to the freeze-dried toehold switch, it becomes rehydrated. During rehydration, the toehold switches can interact with the trigger oncoprotein or RNA if present, activate translation, and produce a signal. In one embodiment, the signal is a colorimetric response. In another embodiment, the signal is a fluorescent response. The toehold switch diagnoses with visual signal read-out upon a color change. If the trigger RNA is not present in the sample, indicating the targeted infection or disease is not present in the subject, no signal will be generated. A fluid sample can be introduced to the toehold switches, also referred to as the synthetic gene network. If the trigger or target RNA is present within the sample, the patch, or a portion thereof, will change color over a time frame of about thirty to sixty minutes. This example time of up to one hour for test results is significantly quicker and easier than sending the sample out to a lab for testing. This can be particularly important in environments where lab facilities are not convenient or available. The patient can simply leave the patch on during a clinical visit for an hour and obtain results within the same day, allowing for improved triaging and treatment options.

### V. Exemplary Dermal Patch Constructions and Layouts

The present invention provides sample fluid collection via microneedles located on the anterior (dermis-facing) side of the patch, generally adjacent to, and in fluid communication with, a sampling chamber, which in turn can be optionally fluidly connected to a detection nanosensing chamber.

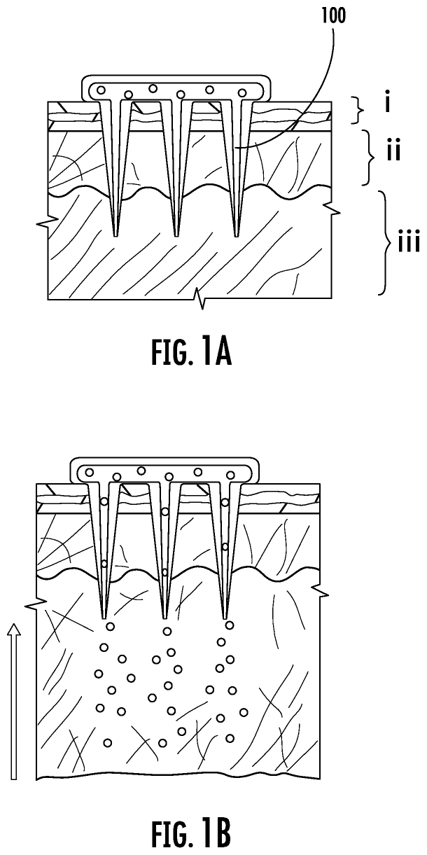

In one embodiment, the microneedles are hollow microneedles inserted into the dermal layers of the skin with interactions through the stratum corneum, the epidermis, and the dermis. Hollow microneedles can be made from suitable materials for biologic environments like silicon or medical-grade stainless steel. FIG. 1 shows hollow microneedles inserted into the skin. In one embodiment, fluid flow occurs via passive or lateral flow transport in which fluid, including miRNAs, will be taken up from an area of high concentration (subject or patient body) to an area of low concentration (dermal patch) and accumulate in a sampling chamber located parallel to the microneedles. The sample then passively flows into detection microwell(s) for interaction with the toehold switches. In another embodiment, sample flow can be aided by a push-valve or an electric current.

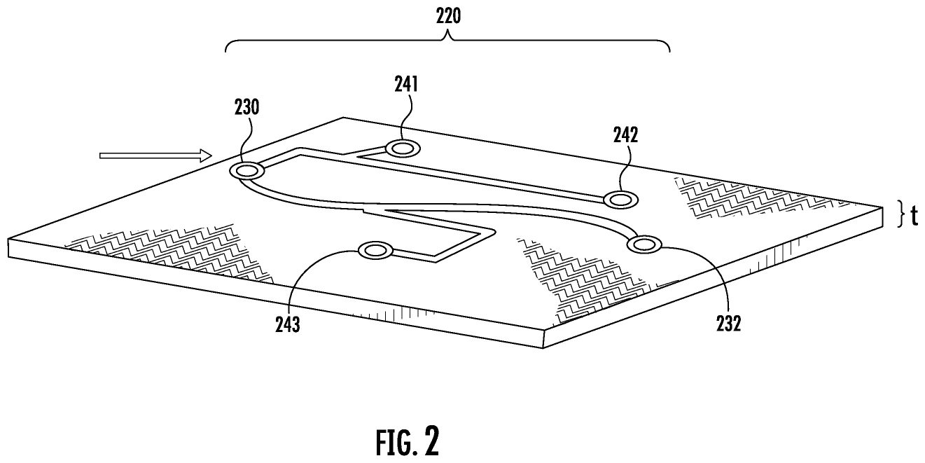

As shown in FIG. 2, other embodiments of the dermal patch features more than one detection microwell (241, 242, 243) in a single patch, allowing the detection of more than one target miRNA sequence. For example, a single patch could include multiple types of toehold switches customized to react to a variety of trigger RNAs. Effectively, a single patch could detect any of several diseases. In some embodiments, the dermal patch can have different signals for each of the diseases it was capable of detecting.

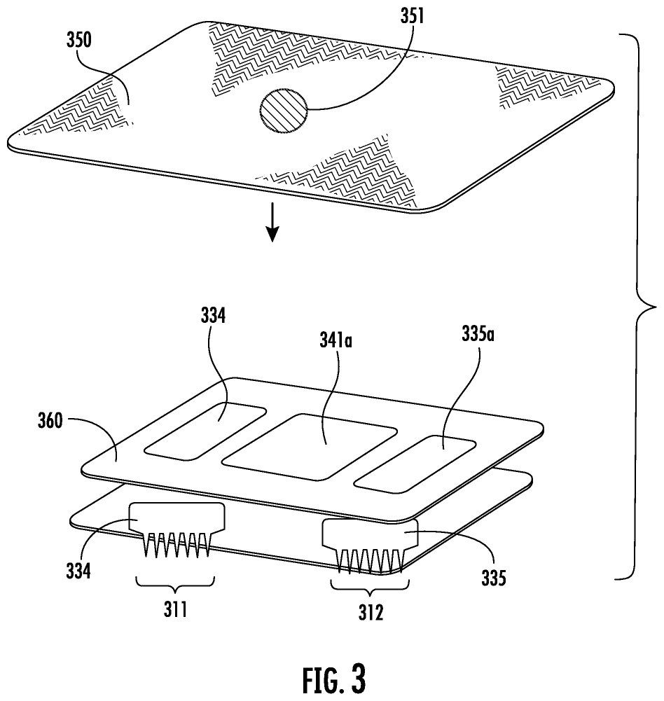

In one embodiment, as shown in FIG. 3, the dermal patch includes a single detection microwell (upper view, 341a) and two microneedle arrays (311, 312). The single detection embodiment detects at least one trigger analyte associated with gynecologic diseases. The dermal patch outermost layer (350) protects the middle layer and the space where the detection signal will be observed. The materials used in the protective layer may be a non-woven polyester or flexible film polyethylene, or other proadhesives. As shown, the posterior layer (350) extends beyond the microfluidic layer (360) and can include an adhesive that holds the patch to the subject. In certain embodiments, an adhesive that will hold the dermal patch to the skin of the subject without damaging or irritating the skin is preferred. Exemplary patch dimensions are 5×5 cm and 1 mm in thickness, but other embodiments of the patch can be different sizes or shapes.

In this embodiment, the detection microwell is in the middle microfluidic layer (360) and resides in fluid communication with the microneedle arrays (311, 312) that extract the sample fluid. When the sample flows into the detection microwell, a detection signal is emitted if the trigger RNA is present. The detection signal is viewable through single detection window (351) wherein the detection signal is only observed when the trigger RNA correlating to the disease of interest is recognized by the toehold sequence. The detection signal is visible through the posterior layer (350) and easily observed by the subject, or a medical professional.

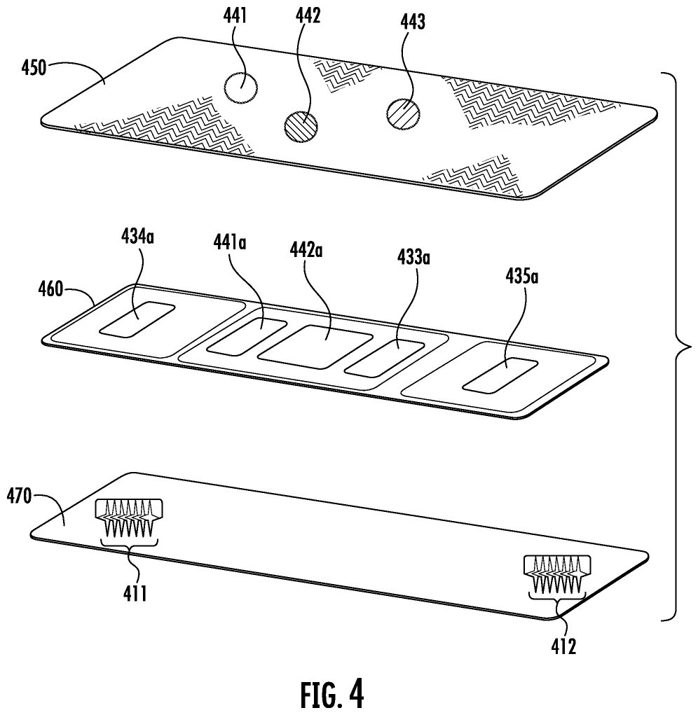

In another embodiment, the dermal patch has multiple detection microwells. One such multiple diagnostic embodiment is shown in FIG. 4 and would function in a similar way to a single diagnostic embodiment, but have several detection microwells to test for the presence of more than one trigger analyte at a time. In this embodiment, the dimensions of the patch are 5×8 cm and 1 mm in thickness, but the patch can be different sizes or shapes.

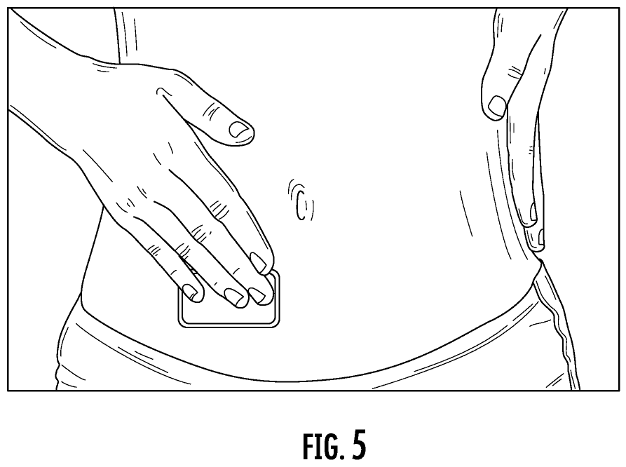

In a preferred embodiment, this patch is worn on the pelvic region of the patient, for best localization of the trigger analytes indicative of gynecological malignancies, as shown in FIG. 5.

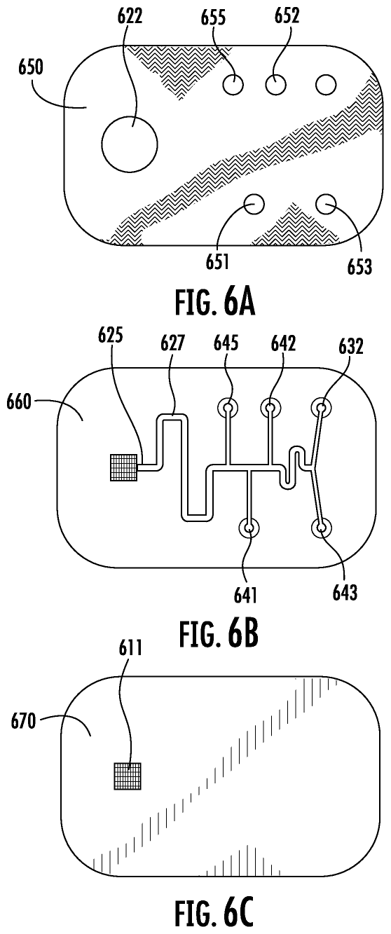

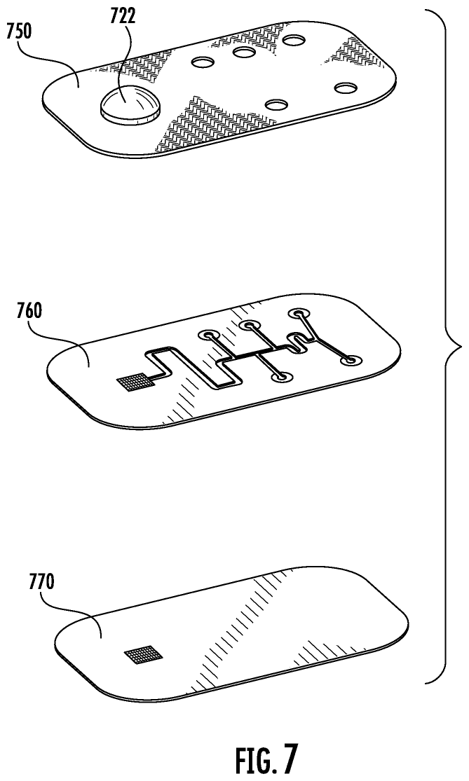

Another embodiment of a diagnostic patch is shown in FIG. 6. FIG. 6 shows top views of each layer of a diagnostic dermal patch having 3 layers: a posterior exterior layer (FIG. 6A), a microfluidic layer (FIG. 6B) disposed between the posterior (visible after application) layer and the anterior (skin-facing) layer, and an anterior layer (FIG. 6C). The layers can be manufactured separately or integrally. The relative position of the layers in an exemplary assembly of separate layers is shown in FIG. 7. Each layer can independently provide structural support and/or protection of the microneedle or microfluidic components.

In FIG. 6A, the posterior layer (650) has indicators and windows to guide the user in operating the dermal patch. A microneedle activation indicator (622), which can be a printed or raised shape (e.g., a push button) (722), indicates where to apply pressure to activate the microneedle array. The posterior layer (650) also features multiple windows for viewing signals from the microwells housed in the microfluidic layer (660). Detection windows (651, 652, 653) overlie detection microwells (641, 642, 643), and control window (655) overlies control microwell (645).

In FIG. 6B, the microfluidic layer (660) houses a microchannel (627) connecting a sample inlet (625) to multiple microwells. Each of the detection microwells (641, 642, 643) contains freeze-dried detection reagent(s). One of the microwells can serve as a control microwell (645); it contains a control reagent to confirm the successful uptake of biological fluid. This microfluidic layer (660) also contains a storage well (632) to collect any excess sample volume not required for the detection and control microwells. In this case, the microwells are shown all of a similar shape and dimension, but it would be understood that the shape, size, position, and placement of each of the microwells (detection, control, and collection, as well as sampling chamber, if present) can be independently selected.

The anterior layer (670) shown in FIG. 6C contacts the epidermis of the subject or patient when in use. The microneedle array (611) is housed in the anterior layer (670). The microneedle array can alternatively be housed in another layer as long as fluidic connection to the sample inlet on the posterior side of the array and access to the skin on the anterior side of the array are maintained. Adhesive to promote and maintain contact of the dermal patch to the skin upon application can be included on the anterior face of the anterior layer, or on the anterior face of an overhanging portion of a posterior layer. The anterior layer can also include a microneedle shield (not shown), which protects the microneedle array from environmental contaminants until activated. This microneedle shield can be, for example, a removeable shield that is removed before applying the patch to the skin or a pierceable shield that remains on when applying the patch and is penetrated upon activation of the microneedle array.

### EXAMPLES

### Example 1: Lymph Node Depth and Concentration in Female Porcine Hides

The hides of 6-month-old female pigs were analyzed to characterize tissue layer thicknesses and the lymph node (LN) environment. Hides were 10 by 12 inches in area. Tissue layer thickness (Table 2) and the number of lymph nodes (Table 3) were recorded for four regions of the hides (i.e., lateral left, medial left, medial right, and lateral right) for each tissue layer including the stratum corneum, stratum lucidum, stratum granulosum, and stratum spinosum. Tissue layer thickness were measured using calipers.

TABLE 2
Tissue thickness for different regions and layers of the porcine
epidermis. Standard deviations are designated in parentheses.

| | Thickness (mm) | | | |
|---|---|---|---|---|
| | Lateral Left | Medial Left | Medial Right | Lateral Right |
| stratum corneum (n = 3) | 3.54 (1.71) | 4.35 (1.96) | 4.07 (1.19) | 4.38 (1.31) |
| stratum lucidum (n = 4) | 6.97 (2.16) | 8.84 (4.08) | 5.58 (2.83) | 7.83 (2.60) |
| stratum granulosum (n = 4) | 11.47 (2.26) | 16.75 (2.26) | 14.125 (0.94) | 12.69 (1.79) |
| stratum spinosum (n = 4) | 18.84 (7.67) | 22.90 (4.02) | 21.77 (1.18) | 17.79 (2.04) |

TABLE 3
Number of lymph nodes for different regions and layers of the porcine
epidermis. Standard deviations are designated in parentheses.

| | Number of Lymph Nodes | | | |
|---|---|---|---|---|
| | Lateral Left | Medial Left | Medial Right | Lateral Right |
| stratum lucidum (n = 4) | 15 (10) | 15 (10) | 20 (6) | 14 (6) |
| stratum granulosum (n = 4) | 18 (2) | 25 (3) | 16 (9) | 14 (5) |
| stratum spinosum (n = 4) | 16 (5) | 15 (8) | 17 (8) | 18 (2) |

### Example 2: Microneedle-Mediated Dermal Patch Collection of Dye Fluid from the Lymph Nodes from Female Porcine Hides

Frozen stratum corneum layers were thawed and rehydrated with PBS. Lymph nodes (LNs) were injected with 10 μL of methylene blue (MB) or PBS (controls). 19×19 Gantrez hydrogel microneedle arrays (Ripolin A et al., International Journal of Pharmaceutics (2017) 521:92-101) were applied to the interior side of the stratum corneum directly over a LN for 5, 15, 30, 45 min, 6 h, or 24 h to collect lymphatic and interstitial fluid. Microneedle arrays were weighed before and after microneedle array application to determine the change in mass. Using the calculated weight difference before and after microneedle array application and the density of lymphatic fluid (1 g/mL), the extracted sample volume could be estimated (Table 4).

Lymphatic fluid and either MB or PBS were extracted from the microneedle arrays with two washing steps. For each wash, the microneedle array was added to a 15 mL tube with 500 μL of TRIzol reagent and then shaken for 30 s. The extracted media and wash solution were collected after each wash and then pooled for analysis. For microneedles that were applied to LNs injected with MB, the absorbance of the recovered dye was measured via NanoDrop. Three microneedle arrays were applied at each time point. Microneedle arrays were applied to control, PBS injected LNs for only 5 min.

TABLE 4
Estimated lymphatic fluid volume extracted from microneedles
after various application times (5 min-24 h). The extraction
fluid volume estimates were calculated based on the density
of interstitial fluid and the difference in microneedle
mass pre- and post-microneedle array application.

| Application Time | | Estimated Extracted Fluid Volume (μL) | Range (μL) |
|---|---|---|---|
| 5 | min* | 60.0 (10.0) | 50-70 |
| 5 | min | 56.7 (11.5) | 50-70 |
| 15 | min | 86.7 (37.9) | 60-130 |
| 30 | min | 100.0 (45.8) | 50-140 |
| 45 | min | 170.0 (60.0) | 110-230 |
| 6 | h** | 90.0 (70.7) | 40-140 |
| 24 | h | 893.3 (113.7) | 800-1020 |

*Control LN injected with 10 μL of PBS. The remaining samples were injected with 10 μL of MB.
**n = 2. The remaining time points had 3 samples.

Although the foregoing invention has been described in some detail by way of illustration and example for purposes of clarity of understanding, one of skill in the art will appreciate that certain changes and modifications may be practiced within the scope of the appended claims. Although particular features may be described herein with respect to certain embodiments, such features may be applied to any embodiment of the present invention. In addition, each reference provided herein is incorporated by reference in its entirety to the same extent as if each reference was individually incorporated by reference.

What is claimed is:

1. A method for collecting a biological fluid sample, the method comprising:
applying a dermal patch comprising a microneedle array to a skin location in proximity to at least one lymph node in a subject or patient;
contacting the microneedle array intradermally to access a biological fluid;
allowing the microneedle array to take up a biological fluid sample;
collecting the biological fluid sample into a sampling chamber housed within the dermal patch.

2. The method of claim 1, further comprising activating the microneedle array by applying pressure or electric current.

3. The method of claim 1, wherein the lymph node is a cervical, clavical, pectoral, axillary, brachial, mediastinal, hilar, spleen, paraaortic, mesenteric, iliac, inguinal, femoral, or popliteal lymph node.

4. The method of claim 3, wherein the lymph node is a paraaortic, mesenteric, or iliac lymph node.

5. The method of claim 1, wherein the subject or patient is female.

6. The method of claim 1, further comprising detecting a trigger analyte in the biological fluid sample ex vivo or in situ.

7. The method of claim 6, wherein:
the skin location is a pelvic region of the subject or patient; and
the trigger analyte correlates to cervical cancer, ovarian cancer, endometriosis-associated ovarian cancer, endometrial cancer, endometriosis, or fibroids.

8. The method of claim 6, wherein:
the skin location is an underarm region of the subject or patient; and
the trigger analyte correlates to breast cancer.

9. The method of claim 6, wherein detecting a trigger analyte comprises: PCR, ELISA, LAMP, or synthetic riboregulator detection.

10. The method of claim 6, wherein the detection step is in situ and further comprises emitting a colorimetric or fluorescent detection signal when a trigger analyte is detected in the biological fluid sample.

11. A dermal patch for use in detecting trigger analytes in a biological fluid sample, comprising:
at least one microneedle array dimensioned to access a biological fluid at a location in proximity to at least one lymph node;
a sample inlet in fluid communication to the microneedle array;
at least one microchannel positioned to provide fluid communication between the sample inlet and at least one detection microwell,
wherein each detection microwell comprises a detection reagent configured to activate and emit a detection signal when the detection reagent contacts a trigger analyte,
and wherein the trigger analyte correlates to the lymph node.

12. The dermal patch of claim 11, wherein the microneedle array comprises hydrogel microneedles.

13. The dermal patch of claim 11, wherein the dermal patch accepts a sample volume of 10-1000 μL.

14. The dermal patch of claim 11, wherein the detection reagent comprises a synthetic riboregulator.

15. The dermal patch of claim 11, further comprising a control microwell, wherein the control microwell comprises a control reagent configured to activate and emit a control signal when the control reagent contacts a biological fluid.

16. The dermal patch of claim 15, wherein the detection signal and the control signal are independently colorimetric or fluorescent.

17. The dermal patch of claim 11, wherein the trigger analyte is an oncoprotein or miRNA.

18. The dermal patch of claim 11, wherein the trigger analyte is correlated to cervical cancer, ovarian cancer, endometriosis-associated ovarian cancer, endometrial cancer, endometriosis, or fibroids.
